(19) United States
(12) Patent Application Publication
(10) Pub. No.: US 2020/0085049 A1
(43) Pub. Date: Mar. 19, 2020

(54) Synergistic Mixtures for Fungal Control in Vegetables

(71) Applicant: Dow AgroSciences LLC, Indianapolis, IN (US)

(72) Inventors: Chenglin Yao, Westfield, IN (US); John T Mathieson, Indianapolis, IN (US)

(73) Assignee: Dow AgroSciences LLC, Indianapolis, IN (US)

(21) Appl. No.: 16/610,124

(22) PCT Filed: May 2, 2018
(86) PCT No.: PCT/US2018/030560

§ 371 Date: Nov. 1, 2019

Related U.S. Application Data
(60) Provisional application No. 62/500,186, filed on May 2, 2017.

(51) Int. Cl. A01N 43/40; C07C 229/08

(52) U.S. Cl. CPC A01N 43/40; C07C 229/08

(57) ABSTRACT

A fungicidal composition containing a fungicidally effective amount of the compound of Formula I, (S)-1,1-bis(4-fluorophenyl)propan-2-yl (3-acetoxy-4-methoxypicolinoyl)-L-alaninate, and at least one fungicide selected from the group consisting of epoxiconazole, prothioconazole, difenoconazole, azoxystrobin, pyraclostrobin, picoxoystrobin, fluxapyroxad, benzovindiflupyr, penthiopyrad, bixafen, chlorothalonil, and mancozeb provides synergistic control of selected fungi.

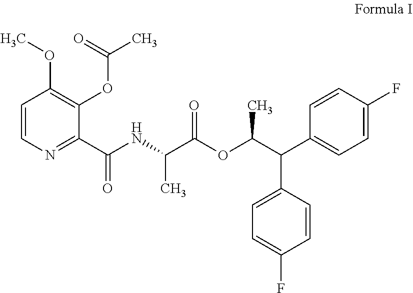 [SMILES: COc1ccnc(C(=O)N[C@@H](C)C(=O)O[C@@H](C)C(c2ccc(F)cc2)c2ccc(F)cc2)c1OC(C)=O]

### CROSS REFERENCE TO RELATED APPLICATIONS

This is a national phase entry under 35 U.S.C. § 371 of international patent application PCT/US18/030560, filed on May 2, 2018 and published in English as international patent publication WO2018204437 on Nov. 8, 2018, which claims the benefit of U.S. Provisional Patent Application Ser. No. 62/500,186 filed May 2, 2017, which is expressly incorporated by reference herein.

### FIELD

This disclosure concerns a synergistic fungicidal composition containing (a) the compound of Formula I and (b) at least one fungicide selected from the group consisting of a sterol biosynthesis inhibitor, for example prothioconazole, epoxiconazole, cyproconazole, myclobutanil, metconazole, difenoconazole, tebuconazole, tetraconazole, fenbuconazole, propiconazole, fluquinconazole, flusilazole, and flutriafol; a strobilurin, for example pyraclostrobin, fluoxastrobin, azoxystrobin, trifloxystrobin, picoxystrobin, and kresoxim methyl; a succinate dehydrogenase inhibitor, for example fluxapyroxad, benzovindiflupyr, penthiopyrad, isopyrazam, bixafen, boscalid, penflufen, and fluopyram; a multi-site inhibitor, for example mancozeb and chlorothalonil, or other commercial fungicides to provide control of any plant fungal pathogen.

### BACKGROUND AND SUMMARY

Fungicides are compounds, of natural or synthetic origin, which act to protect plants against damage caused by fungi. Current methods of agriculture rely heavily on the use of fungicides. In fact, some crops cannot be grown usefully without the use of fungicides. Using fungicides allows a grower to increase the yield and the quality of the crop, and consequently, increase the value of the crop. In most situations, the increase in value of the crop is worth at least three times the cost of the use of the fungicide.

However, no one fungicide is useful in all situations and repeated usage of a single fungicide frequently leads to the development of resistance to that and related fungicides. Consequently, research is being conducted to produce fungicides and combinations of fungicides that are safer, that have better performance, that require lower dosages, that are easier to use, and that cost less.

Synergism occurs when the activity of two or more compounds exceeds the activities of the compounds when used alone.

It is an object of this disclosure to provide synergistic compositions comprising fungicidal compounds. It is a further object of this disclosure to provide processes that use these synergistic compositions. The synergistic compositions are capable of preventing or curing, or both, diseases caused by fungi of the classes Ascomycetes and Basidiomycetes. In addition, the synergistic compositions have improved efficacy against the Ascomycete and Basidiomycete pathogens, including tomato early blight. In accordance with this disclosure, synergistic compositions are provided along with methods for their use.

### DETAILED DESCRIPTION

The present disclosure concerns a synergistic fungicidal mixture comprising a fungicidally effective amount of (a) the compound of Formula I and (b) at least one fungicide selected from the compounds of the following groups A.1, B.1 and C.1:

A.1 Sterol biosynthesis inhibitors (SBI fungicides) selected from the following groups a), b) and c):

a) C14 demethylase inhibitors (DMI fungicides), for example prothioconazole, epoxiconazole, cyproconazole, myclobutanil, metconazole, difenoconazole, tebuconazole, tetraconazole, fenbuconazole, propiconazole, fluquinconazole, flusilazole, flutriafol and prochloraz;

b) Delta 14-reductase inhibitors, for example, fenpropimorph and aldimorph;

c) Inhibitors of 3-keto reductase such as fenhexamid;

B.1 Respiration inhibitors selected from the following groups a) and b):

a) inhibitors of complex II (SDHI fungicides, e.g. carboxamides), for example fluxapyroxad, benzovindiflupyr, penthiopyrad, isopyrazam, bixafen, boscalid, penflufen, and fluopyram;

b) inhibitors of complex III at the Q, site (e.g. strobilurins), for example pyraclostrobin, fluoxastrobin, azoxystrobin, trifloxystrobin, picoxystrobin, and kresoxim methyl;

C.1 Inhibitors with multi-site action selected from the following groups a) and b):

a) thio- and dithiocarbamates, such as mancozeb;

b) organochlorine compounds (e.g. phthalimides, sulfamides, chloronitriles) such as chlorothalonil;

or other commercial fungicides to provide control of any plant fungal pathogen.

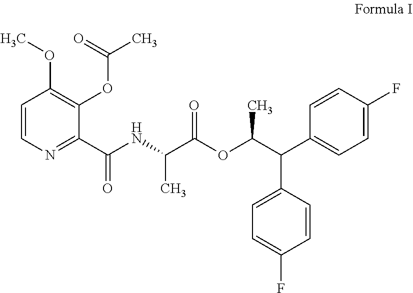 [SMILES: COc1ccnc(C(=O)N[C@@H](C)C(=O)O[C@@H](C)C(c2ccc(F)cc2)c2ccc(F)cc2)c1OC(C)=O]

As used herein, the compound of Formula I is (S)-1,1-bis(4-fluorophenyl)propan-2-yl (3-acetoxy-4-methoxypicolinoyl)-L-alaninate. The compound of Formula I provides control of a variety of pathogens in economically important crops including, but not limited to, the causal agent of tomato early blight, Alternaria solani (ALTESO).

As used herein, epoxiconazole is the common name for (2RS,3SR)-1-[3-(2-chlorophenyl)-2,3-epoxy-2-(4-fluorophenyl)propyl]-1H-1,2,4-triazole and possesses the following structure:

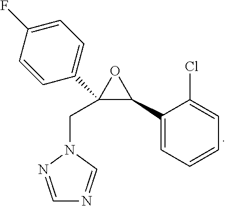 [SMILES: Fc1ccc([C@]2(Cn3cncn3)O[C@H]2c2ccccc2Cl)cc1]

Its fungicidal activity is described in The Pesticide Manual, Fifteenth Edition, 2009. Epoxiconazole provides broad spectrum control, with preventive and curative action, of diseases caused by Ascomycetes, Basidiomycetes and Deuteromycetes in bananas, cereals, coffee, rice and sugar beet.

As used herein, prothioconazole is the common name for 2-[(2RS)-2-(1-chlorocyclopropyl)-3-(2-chlorophenyl)-2-hydroxypropyl]-2H-1,2,4-triazole-3 (4H)-thione and possesses the following structure:

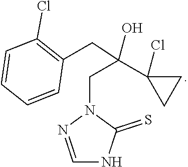 [SMILES: OC(Cc1ccccc1Cl)(Cn1nc[nH]c1=S)C1(Cl)CC1]

Its fungicidal activity is described in The Pesticide Manual, Fifteenth Edition, 2009. Prothioconazole provides control of diseases such as eyespot (Pseudocercosporella herpotrichoides), Fusarium ear blight (Fusarium spp., Microdochium nivale), leaf blotch diseases (Zymoseptoria tritici, Parastagonospora nodorum, Pyrenophora spp., Rhynchosporium secalis, etc.), rust (Puccinia spp.) and powdery mildew (Blumeria graminis), by foliar application, in wheat, barley and other crops.

As used herein, difenoconazole is the common name for 3-chloro-4-[(2RS, 4RS, 2RS, 4RS)-4-methyl-2-(1H-1,2,4-triazol-1-ylmethyl)-1,3-dioxolan-2-yl]phenyl 4-chlorophenyl ether and possesses the following structure:

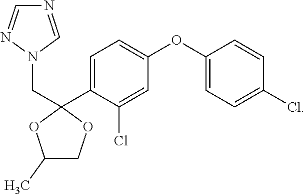 [SMILES: CC1COC(Cn2cncn2)(c2ccc(Oc3ccc(Cl)cc3)cc2Cl)O1]

Its fungicidal activity is described in BCPC Online Pesticide Manual—Latest Version. Difenoconazole provides broad spectrum control, with preventive and curative action, of diseases caused by Ascomycetes, Basidiomycetes and Deuteromycetes in grapes, pome fruit, stone fruit, potatoes, sugar beet, oilseed rape, bananas, cereals, rice, soybeans, ornamentals and various vegetable crops.

As used herein, azoxystrobin is the common name for (E)-2-{2-[6-(2-cyanophenoxy)pyrimidin-4-yloxy]phenyl}-3-methoxyacrylate and possesses the following structure:

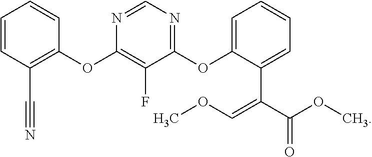 [SMILES: CO/C=C(/C(=O)OC)c1ccccc1Oc1ncnc(Oc2ccccc2C#N)c1F]

Its fungicidal activity is exemplified in The e-Pesticide Manual, Version 5.2, 2011. Exemplary uses of azoxystrobin include, but are not limited to, control of the following pathogens: Erysiphe graminis, Puccinia spp., Parastagonospora nodorum, Zymoseptoria tritici and Pyrenophora teres on temperate cereals; Pyricularia oryzae and Rhizoctonia solani on rice; Plasmopara viticola and Uncinula necator on vines; Sphaerotheca fuliginea and Pseudoperonospora cubensis on cucurbitaceae; Phytophthora infestans and Alternaria solani on potato and tomato; Mycosphaerella arachidis, Rhizoctonia solani and Sclerotium rolfsii on peanut; Monilinia spp. and Cladosporium carpophilum on peach; Pythium spp. and Rhizoctonia solani on turf; Mycosphaerella spp. on banana; Cladosporium caryigenum on pecan; Elsinoe fawcettii, Colletotrichum spp. and Guignardia citricarpa on citrus; Colletotrichum spp. and Hemileia vastatrix on coffee.

As used herein, pyraclostrobin is the common name for methyl N-[2-[[[1-(4-chlorophenyl)-1H-pyrazol-3-yl]oxy]methyl]phenyl]-N-methoxycarbamate and possesses the following structure:

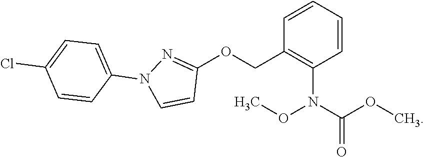 [SMILES: COC(=O)N(OC)c1ccccc1COc1ccn(-c2ccc(Cl)cc2)n1]

Its fungicidal activity is described in BCPC Online Pesticide Manual—Latest Version. Exemplary uses of pyraclostrobin include, but are not limited to, broad spectrum disease control of major plant pathogens, including Zymoseptoria tritici, Puccinia spp., Drechslera tritici-repentis, Pyrenophora teres, Rhynchosporium secalis and Septoria nodorum in cereals; Mycosphaerella spp. in peanuts; Septoria glycines, Cercospora kikuchii and Phakopsora pachyrhizi in soybeans; Plasmopara viticola and Erysiphe necator in grapes; Phytophthora infestans and Alternaria solani in potatoes and tomatoes; Sphaerotheca fuliginea and Pseudoperonospora cubensis in cucumber; Mycosphaerella fijiensis in bananas; Elsinoe fawcettii and Guignardia citricarpa in citrus and Rhizoctonia solani and Pythium aphanidermatum in turf.

As used herein, picoxystrobin is the common name for methyl (E)-3-methoxy-2-[2-(6-trifluoromethyl-2-pyridyloxymethyl)phenyl]acrylate and possesses the following structure:

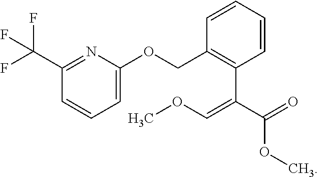 [SMILES: CO/C=C(/C(=O)OC)c1ccccc1COc1cccc(C(F)(F)F)n1]

Its fungicidal activity is described in The e-Pesticide Manual, Version 5.2, 2011. Exemplary uses of picoxystrobin include, but are not limited to, broad-spectrum disease control in cereals, including Mycosphaerella graminicola, Phaeosphaeria nodorum, Puccinia recondita (brown rust), Helminthosporium tritici-repentis (tan spot) and Blumeria graminis fsp. tritici (strobilurin-sensitive powdery mildew) in wheat; Helminthosporium teres (net blotch), Rhynchosporium secalis, Puccinia horde/(brown rust) and Erysiphe graminis fsp. horde/(strobilurin-sensitive powdery mildew) in barley; Puccinia coronata and Helminthosporium avenae in oats; and Puccinia recondita and Rhynchosporium secalis in rye.

As used herein, fluxapyroxad is the common name for 3-(difluoromethyl)-1-methyl-N-(3′,4′,5′-trifluorobiphenyl-2-yl)pyrazole-4-carboxamide and possesses the following structure:

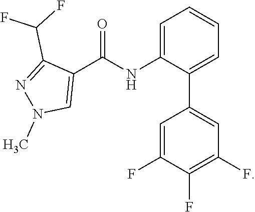 [SMILES: Cn1cc(C(=O)Nc2ccccc2-c2cc(F)c(F)c(F)c2)c(C(F)F)n1]

Its fungicidal activity is exemplified in Agrow Intelligence (https://www.agra-net.net/agra/agrow/databases/agrow-intelligence/). Exemplary uses of fluxapyroxad include, but are not limited to, the control of plant pathogens, such as Helminthosporium teres (net blotch), Rhynchosporium secalis (leaf scald), Puccinia horde/(brown rust), and Erysiphe graminis fsp. horde/(powdery mildew) in a range of crops, such as barley, maize, and soybeans.

As used herein, benzovindiflupyr is the common name for N-[(1RS,4SR)-9-(dichloromethylene)-1,2,3,4-tetrahydro-1,4-methanonaphthalen-5-yl]-3-(difluoromethyl)-1-methylpyrazole-4-carboxamide and possesses the following structure:

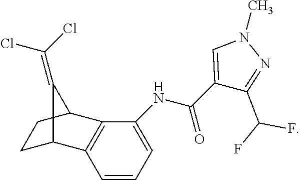 [SMILES: Cn1cc(C(=O)Nc2cccc3c2C2CCC3C2=C(Cl)Cl)c(C(F)F)n1]

Its fungicidal activity is exemplified in Agrow Intelligence (https://www.agra-net.net/agra/agrow/databases/agrow-intelligence/). Exemplary uses of benzovindiflupyr include, but are not limited to, controlling a variety of pathogens such as Botrytis spp., Erysiphe spp., Rhizoctonia spp., Septoria spp., Phytophthora spp., Pythium spp., Phakopsora pachyrhizi, and Puccinia recondita, in a range of crops including vines, cereals, soybeans, cotton, and fruit and vegetable crops.

As used herein, penthiopyrad is the common name for N-[2-(1,3-dimethylbutyl)-3-thienyl]-1-methyl-3-(trifluoromethyl)-1H-pyrazole-4-carboxamide and possesses the following structure:

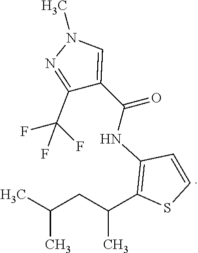 [SMILES: CC(C)CC(C)c1sccc1NC(=O)c1cn(C)nc1C(F)(F)F]

Its fungicidal activity is described in The Pesticide Manual, Fourteenth Edition, 2006. Penthiopyrad provides control of rust and Rhizoctonia diseases, as well as grey mold, powdery mildew and apple scab.

As used herein, bixafen is the common name for N-(3′,4′-dichloro-5-fluoro[1,1′-biphenyl]-2-yl)-3-(difluoromethyl)-1-methyl-1H-pyrazole-4-carboxamide and possesses the following structure:

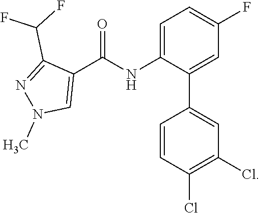 [SMILES: Cn1cc(C(=O)Nc2ccc(F)cc2-c2ccc(Cl)c(Cl)c2)c(C(F)F)n1]

Its fungicidal activity is described in BCPC Online Pesticide Manual—Latest Version. Exemplary uses of bixafen include, but are not limited to, broad-spectrum disease control in cereals, including Zymoseptoria tritici, Puccinia triticina, Puccinia striiformis, Oculimacula spp. and Pyrenophora tritici-repentis in wheat and against Pyrenophora teres, Ramularia collo-cygni, Rhynchosporium secalis and Puccinia hordei in barley.

As used herein, chlorothalonil is the common name for tetrachloroisophthal-onitrile and possesses the following structure:

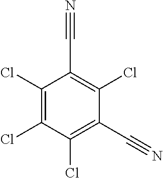 [SMILES: N#Cc1c(Cl)c(Cl)c(Cl)c(C#N)c1Cl]

Its fungicidal activity is described in The Pesticide Manual, Fifteenth Edition, 2009. Chlorothalonil provides control of many fungal diseases in a wide range of crops, including pome fruit, stone fruit, almonds, citrus fruit, bush and cane fruit, cranberries, strawberries, pawpaws, bananas, mangoes, coconut palms, oil palms, rubber, pepper, vines, hops, vegetables, cucurbits, tobacco, coffee, tea, rice, soya beans, peanuts, potatoes, sugar beet, cotton, maize, ornamentals, mushrooms, and turf.

As used herein, mancozeb is the common name for [[2-[(dithiocarboxy)amino]ethyl]carbamodithioato(2-)-κS,κS′]manganese mixture with [[2-[(dithiocarboxy)amino]ethyl]carbamodithioato(2-)-κS,κS′]zinc and possesses the following structure:

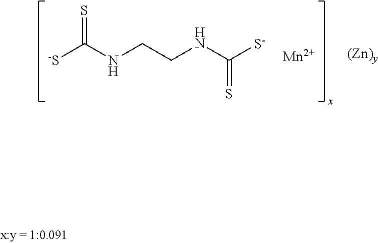

Its fungicidal activity is described in The Pesticide Manual, Fifteenth Edition, 2009. Mancozeb provides control of a wide range of fungal pathogens on a variety of fruits, vegetables and field crops.

In the compositions described herein, the concentration ratio of the mixture of the compound of Formula I to other fungicides at which the fungicidal effect is synergistic against tomato early blight caused by Alternaria solani (ALTESO) in protectant applications lies within the range from about 50:1 to about 1:20.

In the compositions described herein, the concentration ratio of the mixture of the compound of Formula I to a sterol biosynthesis inhibitor at which the fungicidal effect is synergistic against ALTESO in protectant applications lies within the range from about 10:1 to about 1:1.6. In one embodiment, the concentration ratio of the mixture of the compound of Formula I to epoxiconazole at which the fungicidal effect is synergistic against ALTESO in protectant applications is from about 10:1 to about 1:1.6. In another embodiment, the concentration ratio of the mixture of the compound of Formula I to prothioconazole at which the fungicidal effect is synergistic against ALTESO in protectant applications is from about 10:1 to about 1.25:1. In yet another embodiment, the concentration ratio of the mixture of the compound of Formula I to difenoconazole at which the fungicidal effect is synergistic against ALTESO in protectant applications is from about 5:1 to about 1:1.6.

In the compositions described herein, the concentration ratio of the mixture of the compound of Formula I to a strobilurin fungicide at which the fungicidal effect is synergistic against ALTESO in protectant applications lies within the range from about 50:1 to about 1:1.6. In one embodiment, the concentration ratio of the mixture of the compound of Formula I to azoxystrobin at which the fungicidal effect is synergistic against ALTESO in protectant applications lies within the range from about 50:1 to about 3:1. In another embodiment, the concentration ratio of the mixture of the compound of Formula I to pyraclostrobin at which the fungicidal effect is synergistic against ALTESO in protectant applications lies within the range from about 50:1 to about 3:1. In yet another embodiment, the concentration ratio of the mixture of the compound of Formula I to picoxystrobin at which the fungicidal effect is synergistic against ALTESO in protectant applications lies within the range from about 10:1 to about 1:1.6.

In the compositions described herein, the concentration ratio of the mixture of the compound of Formula I to a succinate dehydrogenase inhibitor (SDHI) fungicide at which the fungicidal effect is synergistic against ALTESO in protectant applications lies within the range from about 50:1 to about 1:1.6. In one embodiment, the concentration ratio of the mixture of the compound of Formula I to fluxapyroxad at which the fungicidal effect is synergistic against ALTESO in protectant applications lies within the range from about 50:1 to about 3:1. In another embodiment, the concentration ratio of the mixture of the compound of Formula I to benzovindiflupyr at which the fungicidal effect is synergistic against ALTESO in protectant applications is from about 10:1 to about 1:1.6. In other embodiments, the concentration ratio of the mixture of the compound of Formula I to penthiopyrad at which the fungicidal effect is synergistic against ALTESO in protectant applications is from about 10:1 to about 1.3:1. In yet another embodiment, the concentration ratio of the mixture of the compound of Formula I to bixafen at which the fungicidal effect is synergistic against ALTESO in protectant applications is from about 10:1 to about 1:1.6.

In the compositions described herein, the concentration ratio of the mixture of the compound of Formula I to a multi-site inhibitor fungicide at which the fungicidal effect is synergistic against ALTESO in protectant applications lies within the range from about 1:1.3 to about 1:20. In one embodiment, the concentration ratio of the mixture of the compound of Formula I to chlorothalonil at which the fungicidal effect is synergistic against ALTESO in protectant applications lies within the range from about 1:1.3 to about 1:20. In another embodiment, the concentration ratio of the mixture of the compound of Formula I to mancozeb at which the fungicidal effect is synergistic against ALTESO in protectant applications lies within the range from about 1:1.3 to about 1:20.

The rate at which the synergistic composition is applied will depend upon the particular type of fungus to be controlled, the degree of control required and the timing and method of application. In general, the compositions described herein can be applied at an application rate of between about 40 grams per hectare (g/ha) and about 2650 g/ha based on the total amount of active ingredients in the composition.

The compositions comprising the compound of Formula I and a sterol biosynthesis inhibitor can be applied at an application rate of between about 40 g/ha and about 400 g/ha based on the total amount of active ingredients in the composition. Epoxiconazole is applied at a rate of between about 50 g/ha and about 250 g/ha and the compound of Formula I is applied at a rate between about 10 g/ha and about 150 g/ha. Prothioconazole is applied at a rate of between about 50 g/ha and about 250 g/ha and the compound of Formula I is applied at a rate between about 10 g/ha and about 150 g/ha. Difenoconazole is applied at a rate of between about 30 g/ha and about 125 g/ha and the compound of Formula I is applied at a rate between about 10 g/ha and about 150 g/ha.

The compositions comprising the compound of Formula I and a strobilurin fungicide can be applied at an application rate of between about 40 g/ha and about 350 g/ha based on the total amount of active ingredients in the composition. Azoxystrobin is applied at a rate of between about 30 g/ha and about 200 g/ha and the compound of Formula I is applied at a rate between about 10 g/ha and about 150 g/ha. Pyraclostrobin is applied at a rate of between about 30 g/ha and about 200 g/ha and the compound of Formula I is applied at a rate between about 10 g/ha and about 150 g/ha. Picoxystrobin is applied at a rate of between about 30 g/ha and about 200 g/ha and the compound of Formula I is applied at a rate between about 10 g/ha and about 150 g/ha.

The compositions comprising the compound of Formula I and a carboxamide SDHI fungicide can be applied at an application rate of between about 40 g/ha and about 350 g/ha based on the total amount of active ingredients in the composition. Fluxapyroxad is applied at a rate of between about 45 g/ha and about 200 g/ha and the compound of Formula I is applied at a rate between about 10 g/ha and about 150 g/ha. Benzovindiflupyr is applied at a rate of between about 45 g/ha and about 200 g/ha and the compound of Formula I is applied at a rate between about 10 g/ha and about 150 g/ha. Penthiopyrad is applied at a rate of between about 45 g/ha and about 200 g/ha and the compound of Formula I is applied at a rate between about 10 g/ha and about 150 g/ha. Bixafen is applied at a rate of between about 30 g/ha and about 200 g/ha and the compound of Formula I is applied at a rate between about 10 g/ha and about 150 g/ha.

The compositions comprising the compound of Formula I and a multi-site inhibitor can be applied at an application rate of between about 1010 g/ha and about 2650 g/ha based on the total amount of active ingredients in the composition. Chlorothalonil is applied at a rate of between about 1000 g/ha and about 2500 g/ha and the compound of Formula I is applied at a rate between about 10 g/ha and about 150 g/ha. Mancozeb is applied at a rate of between about 1000 g/ha and about 2500 g/ha and the compound of Formula I is applied at a rate between about 10 g/ha and about 150 g/ha.

The components of the synergistic mixture described herein can be applied either separately or as part of a multipart fungicidal system.

The synergistic mixture of the present disclosure can be applied in conjunction with one or more other fungicides to control a wider variety of undesirable diseases. When used in conjunction with other fungicide(s), the presently claimed compounds may be formulated with the other fungicide(s), tank mixed with the other fungicide(s) or applied sequentially with the other fungicide(s). Such other fungicides may include 2-(thiocyanatomethylthio)-benzothiazole, 2-phenylphenol, 8-hydroxyquinoline sulfate, ametoctradin, amisulbrom, antimycin, Ampelomyces quisqualis, azaconazole, Bacillus subtilis, Bacillus subtilis strain QST713, benalaxyl, benomyl, benthiavalicarb-isopropyl, benzylaminobenzene-sulfonate (BABS) salt, bicarbonates, biphenyl, bismerthiazol, bitertanol, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenzoquation, diflumetorim, dimethomorph, dimoxystrobin, diniconazole, diniconazole-M, dinobuton, dinocap, diphenylamine, dipymetitrone, dithianon, dodemorph, dodemorph acetate, dodine, dodine free base, edifenphos, enestrobin, enestroburin, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, fluindapyr, flumorph, fluopicolide, fluopyram, fluoroimide, fluoxastrobin, fluquinconazole, flusilazole, flusulfamide, flutianil, flutolanil, flutriafol, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isofetamide, isoprothiolane, isopyrazam, isotianil, kasugamycin, kasugamycin hydrochloride hydrate, kresoxium-methyl, laminarin, mancopper, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxathiapiprolin, oxine-copper, oxpoconazole fumarate, oxycarboxin, pefurazoate, penconazole, pencycuron, penflufen, pentachlorophenol, pentachlorophenyl laurate, phenylmercury acetate, phosphonic acid, phthalide, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, pydiflumetofen, pyrametostrobin, pyraoxystrobin, pyraziflumid, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)—N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-1-enyl)phenyl thiocyanateme, aminopyrifen, ampropylfos, anilazine, azithiram, barium polysulfide, Bayer 32394, benodanil, benquinox, bentaluron, benzamacril; benzamacril-isobutyl, benzamorf, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, cufraneb, cupric hydrazinium sulfate, cuprobam, cyclafuramid, cypendazole, cyprofuram, decafentin, dichlobentiazox, dichlone, dichlozoline, diclobutrazol, dimethirimol, dinocton, dinosulfon, dinoterbon, dipyrithione, ditalimfos, dodicin, drazoxolon, EBP, ESBP, etaconazole, etem, ethirim, fenaminosulf, fenapanil, fenitropan, fluindapyr, fluopimomide, fluotrimazole, furcarbanil, furconazole, furconazole-cis, furmecyclox, furophanate, glyodine, griseofulvin, halacrinate, Hercules 3944, hexylthiofos, ICIA0858, inpyrfluxam, ipfentrifluconazole, ipflufenoquin, isoflucypram, isopamphos, isovaledione, mandestrobin, mebenil, mecarbinzid, mefentrifluconazole, metazoxolon, methfuroxam, methylmercury dicyandiamide, metsulfovax, metyltetraprole, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, phenylmercury dimethyldithiocarbamate, phenylmercury nitrate, phosdiphen, prothiocarb; prothiocarb hydrochloride, pydiflumetofen, pyracarbolid, pyrapropoyne, pyridachlometyl, pyridinitril, pyroxychlor, pyroxyfur, quinacetol; quinacetol sulfate, quinazamid, quinconazole, quinofumelin, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, urbacid, zarilamid, and any combinations thereof.

The compositions of the present disclosure are preferably applied in the form of a formulation comprising a composition of (a) a compound of Formula I and (b) at least one fungicide selected from the group consisting of epoxiconazole, prothioconazole, difenoconazole, azoxystrobin, pyraclostrobin, picoxystrobin, fluxapyroxad, benzovindiflupyr, picoxystrobin, bixafen, mancozeb, and chlorothalonil, together with a phytologically acceptable carrier.

Concentrated formulations can be dispersed in water, or another liquid, for application, or formulations can be dust-like or granular, which can then be applied without further treatment. The formulations are prepared according to procedures which are conventional in the agricultural chemical art, but which are novel and important because of the presence therein of a synergistic composition.

The formulations that are applied most often are aqueous suspensions or emulsions. Either such water-soluble, water-suspendable, or emulsifiable formulations are solids, usually known as wettable powders, or liquids, usually known as emulsifiable concentrates, aqueous suspensions, or suspension concentrates. The present disclosure contemplates all vehicles by which the synergistic compositions can be formulated for delivery and use as a fungicide.

As will be readily appreciated, any material to which these synergistic compositions can be added may be used, provided they yield the desired utility without significant interference with the activity of these synergistic compositions as antifungal agents.

Wettable powders, which may be compacted to form water-dispersible granules, comprise an intimate mixture of the synergistic composition, a carrier and agriculturally acceptable surfactants. The concentration of the synergistic composition in the wettable powder is usually from about 10% to about 90% by weight, more preferably about 25% to about 75% by weight, based on the total weight of the formulation. In the preparation of wettable powder formulations, the synergistic composition can be compounded with any of the finely divided solids, such as prophyllite, talc, chalk, gypsum, Fuller's earth, bentonite, attapulgite, starch, casein, gluten, montmorillonite clays, diatomaceous earths, purified silicates or the like. In such operations, the finely divided carrier is ground or mixed with the synergistic composition in a volatile organic solvent. Effective surfactants, comprising from about 0.5% to about 10% by weight of the wettable powder, include sulfonated lignins, naphthalenesulfonates, alkylbenzenesulfonates, alkyl sulfates, and non-ionic surfactants, such as ethylene oxide adducts of alkyl phenols.

Emulsifiable concentrates of the synergistic composition comprise a convenient concentration, such as from about 10% to about 50% by weight, in a suitable liquid, based on the total weight of the emulsifiable concentrate formulation. The components of the synergistic compositions, jointly or separately, are dissolved in a carrier, which is either a water-miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used, such as, for example, terpenic solvents, including rosin derivatives, aliphatic ketones, such as cyclohexanone, and complex alcohols, such as 2-ethoxyethanol.

Emulsifiers which can be advantageously employed herein can be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulfonic acids, oil-soluble salts or sulfated polyglycol ethers and appropriate salts of phosphated polyglycol ether.

Representative organic liquids which can be employed in preparing the emulsifiable concentrates of the present disclosure are the aromatic liquids such as xylene, propyl benzene fractions, or mixed naphthalene fractions, mineral oils, substituted aromatic organic liquids such as dioctyl phthalate, kerosene, dialkyl amides of various fatty acids, particularly the dimethyl amides of fatty glycols and glycol derivatives such as the n-butyl ether, ethyl ether or methyl ether of diethylene glycol, and the methyl ether of triethylene glycol. Mixtures of two or more organic liquids are also often suitably employed in the preparation of the emulsifiable concentrate. The preferred organic liquids are xylene, and propyl benzene fractions, with xylene being most preferred. The surface-active dispersing agents are usually employed in liquid formulations and in the amount of from 0.1 to 20 percent by weight of the combined weight of the dispersing agent with the synergistic compositions. The formulations can also contain other compatible additives, for example, plant growth regulators and other biologically active compounds used in agriculture.

Aqueous suspensions comprise suspensions of one or more water-insoluble compounds, dispersed in an aqueous vehicle at a concentration in the range from about 5% to about 70% by weight, based on the total weight of the aqueous suspension formulation. Suspensions are prepared by finely grinding the components of the synergistic combination either together or separately, and vigorously mixing the ground material into a vehicle comprised of water and surfactants chosen from the same types discussed above. Other ingredients, such as inorganic salts and synthetic or natural gums, may also be added to increase the density and viscosity of the aqueous vehicle. It is often most effective to grind and mix at the same time by preparing the aqueous mixture and homogenizing it in an implement such as a sand mill, ball mill, or piston-type homogenizer.

The synergistic composition may also be applied as a granular formulation, which is particularly useful for applications to the soil. Granular formulations usually contain from about 0.5% to about 10% by weight of the compounds, based on the total weight of the granular formulation, dispersed in a carrier which consists entirely or in large part of coarsely divided attapulgite, bentonite, diatomite, clay or a similar inexpensive substance. Such formulations are usually prepared by dissolving the synergistic composition in a suitable solvent and applying it to a granular carrier which has been preformed to the appropriate particle size, in the range of from about 0.5 to about 3 millimeters (mm). Such formulations may also be prepared by making a dough or paste of the carrier and the synergistic composition, and crushing and drying to obtain the desired granular particle.

Dusts containing the synergistic composition are prepared simply by intimately mixing the synergistic composition in powdered form with a suitable dusty agricultural carrier, such as, for example, kaolin clay, ground volcanic rock, and the like. Dusts can suitably contain from about 1% to about 10% by weight of the synergistic composition/carrier combination.

The formulations may contain agriculturally acceptable adjuvant surfactants to enhance deposition, wetting and penetration of the synergistic composition onto the target crop and organism. These adjuvant surfactants may optionally be employed as a component of the formulation or as a tank mix. The amount of adjuvant surfactant will vary from 0.01 percent to 1.0 percent volume/volume (v/v) based on a spray-volume of water, preferably 0.05 to 0.5 percent. Suitable adjuvant surfactants include ethoxylated nonyl phenols, ethoxylated synthetic or natural alcohols, salts of the esters or sulfosuccinic acids, ethoxylated organosilicones, ethoxylated fatty amines and blends of surfactants with mineral or vegetable oils.

The formulations may optionally include combinations that can comprise at least 1% by weight of one or more of the synergistic compositions with another pesticidal compound. Such additional pesticidal compounds may be fungicides, insecticides, nematicides, miticides, arthropodicides, bactericides or combinations thereof that are compatible with the synergistic compositions of the present disclosure in the medium selected for application, and not antagonistic to the activity of the present compounds. Accordingly, in such embodiments the other pesticidal compound is employed as a supplemental toxicant for the same or for a different pesticidal use. The pesticidal compound and the synergistic composition can generally be mixed together in a weight ratio of from 1:100 to 100:1.

The present disclosure includes within its scope methods for the control or prevention of fungal attack. These methods comprise applying to the locus of the fungus, or to a locus in which the infestation is to be prevented (for example applying to tomato plants), a fungicidally effective amount of the synergistic composition. The synergistic composition is suitable for treatment of various plants at fungicidal levels, while exhibiting low phytotoxicity. The synergistic composition is useful in a protectant or eradicant fashion. The synergistic composition is applied by any of a variety of known techniques, either as the synergistic composition or as a formulation comprising the synergistic composition. For example, the synergistic compositions may be applied to the roots, seeds or foliage of plants for the control of various fungi, without damaging the commercial value of the plants. The synergistic composition is applied in the form of any of the generally used formulation types, for example, as solutions, dusts, wettable powders, flowable concentrates, or emulsifiable concentrates. These materials are conveniently applied in various known fashions.

The synergistic composition has been found to have significant fungicidal effect, particularly for agricultural use. The synergistic composition is particularly effective for use with agricultural crops and horticultural plants, or with wood, paint, leather or carpet backing.

In particular, the synergistic composition is effective in controlling a variety of undesirable fungi that infect useful plant crops. The synergistic composition may be used against a variety of Ascomycete and Basidiomycete fungi, including for example the following representative fungi species: barley leaf scald (Rhynchosporium secalis); barley Ramularia leaf spot (Ramularia collo-cygni); barley net blotch (Pyrenophora teres); barley powdery mildew (Blumeria graminis f. sp. hordei); wheat powdery midlew (Blumeria graminis f sp. tritici); wheat brown rust (Puccinia triticina); stripe rust of wheat (Puccinia striiformis); leaf blotch of wheat (Zymoseptoria tritici); glume blotch of wheat (Parastagonospora nodorum); leaf spot of sugar beets (Cercospora beticola); leaf spot of peanut (Mycosphaerella arachidis); cucumber anthracnose (Colletotrichum lagenarium); cucumber powdery mildew (Podosphaera xanthii); watermelon stem gummy blight (Didymella bryoniae); apple scab (Venturia inaequalis); apple powdery mildew (Podosphaera leucotricha); grey mold (Botrytis cinerea); Sclerotinia white mold (Sclerotinia sclerotiorum); grape powdery mildew (Erysiphe necator); rice blast (Pyricularia oryzae); brown rot of stone fruits (Monilinia fructicola) and black sigatoka disease of banana (Mycosphaerella fijiensis). It will be understood by those in the art that the efficacy of the synergistic compositions for one or more of the foregoing fungi establishes the general utility of the synergistic compositions as fungicides.

The synergistic compositions have a broad range of efficacy as a fungicide. The exact amount of the synergistic composition to be applied is dependent not only on the relative amounts of the components, but also on the particular action desired, the fungal species to be controlled, and the stage of growth thereof, as well as the part of the plant or other product to be contacted with the synergistic composition. Thus, formulations containing the synergistic composition may not be equally effective at similar concentrations or against the same fungal species.

The synergistic compositions are effective in use with plants in a disease-inhibiting and phytologically acceptable amount. The term “disease-inhibiting and phytologically acceptable amount” refers to an amount of the synergistic composition that kills or inhibits the plant disease for which control is desired, but is not significantly toxic to the plant. The exact concentration of synergistic composition required varies with the fungal disease to be controlled, the type of formulation employed, the method of application, the particular plant species, climate conditions, and the like.

The present compositions can be applied to fungi or their locus by the use of conventional ground sprayers, granule applicators, and by other conventional means known to those skilled in the art.

The following examples are provided for illustrative purposes and should not be construed as limitations to the disclosure.

### Examples

Evaluation of Protectant Activity of Fungicide Mixtures vs. Tomato Early Blight (Alternaria solani; Bayer Code: ALTESO):

Tomato seedlings (variety Outdoor Girl) were propagated in a soil-less media (Metro Mix™), with each pot having 1 plant, and used in the test when the first set of leaves was fully expanded. Treatments consisted of fungicide compounds epoxiconazole, prothioconazole, difenoconazole, azoxystrobin, pyraclostrobin, picoxystrobin, fluxapyroxad, benzovindiflupyr, penthiopyrad, bixafen, chlorothalonil, and mancozeb either using individually or as two-way mixture with the compound of Formula I.

The compounds were tested as technical grade material formulated in acetone, and spray solutions contained 10% acetone and 100 ppm Triton X-100. Fungicide solutions were applied onto plants using an automated booth sprayer, which utilized two 6218-1/4 JAUPM spray nozzles operating at 20 pounds per square inch (psi) set at opposing angles to cover both leaf surfaces. All sprayed plants were allowed to air dry prior to further handling. Control plants were sprayed in the same manner with the solvent blank.

Test plants were inoculated with an aqueous spore suspension of Alternaria solani 1 day after fungicide treatments (1-day protectant test). After inoculation the plants were kept in 100% relative humidity for two days to permit spores to germinate and infect the leaf. The plants were then transferred to a growth chamber for disease to develop. When the disease was fully developed on untreated plants, disease severity on the seedlings was assessed and activity was represented by percent of leaf area free of ALTESO infection relative to the untreated plants. The percent disease control was calculated using the equation (1−(disease severity on treated plants/disease severity on untreated plants))*100.

Colby's equation was used to determine the fungicidal effects expected from the mixtures. (See Colby, S. R. Calculation of the synergistic and antagonistic response of herbicide combinations. Weeds 1967, 15, 20-22.)

The following equation was used to calculate the expected activity of mixtures containing two active ingredients, A and B:

Expected=A+B−(A×B/100)
A=observed efficacy of active component A at the same concentration as used in the mixture;
B=observed efficacy of active component B at the same concentration as used in the mixture.

Synergistic interactions between compound I and other fungicides were detected in protectant assays vs. ALTESO (Table 1).

TABLE 1
Synergistic Interactions of the Compound of Formula I and Other Fungicides
in a 1-Day Protectant (1 DP) Alternaria solani (ALTESO) Assay.

| | Rates | ALTESO* | | Synergism |
|---|---|---|---|---|
| Composition | (ppm)* | Observed* | Expected* | Factor* |
| Epoxiconazole + Compound I | 4 + 10 | 93.3 | 16.3 | 5.7 |
| Epoxiconazole + Compound I | 2 + 10 | 65.0 | 12.7 | 5.1 |
| Epoxiconazole + Compound I | 1 + 10 | 61.7 | 10.0 | 6.2 |
| Epoxiconazole + Compound I | 4 + 5 | 75.0 | 22.8 | 3.3 |
| Epoxiconazole + Compound I | 2 + 5 | 71.7 | 19.5 | 3.7 |
| Epoxiconazole + Compound I | 1 + 5 | 63.3 | 17.0 | 3.7 |
| Epoxiconazole + Compound I | 4 + 2.5 | 16.7 | 9.8 | 1.7 |
| Epoxiconazole + Compound I | 2 + 2.5 | 23.3 | 5.9 | 3.9 |
| Epoxiconazole + Compound I | 1 + 2.5 | 13.3 | 3.0 | 4.4 |
| Prothioconazole + Compound I | 2 + 10 | 33.3 | 10.0 | 3.3 |
| Prothioconazole + Compound I | 1 + 10 | 26.7 | 19.0 | 1.4 |
| Prothioconazole + Compound I | 4 + 5 | 60.0 | 41.9 | 1.4 |
| Prothioconazole + Compound I | 2 + 5 | 43.3 | 17.0 | 2.5 |
| Prothioconazole + Compound I | 1 + 5 | 56.7 | 25.3 | 2.2 |
| Prothioconazole + Compound I | 2 + 2.5 | 36.7 | 3.0 | 12.2 |
| Prothioconazole + Compound I | 1 + 2.5 | 46.7 | 12.7 | 3.7 |
| Difenoconazole + Compound I | 4 + 10 | 79.0 | 31.0 | 2.5 |
| Difenoconazole + Compound I | 2 + 10 | 50.0 | 34.0 | 1.5 |
| Difenoconazole + Compound I | 4 + 5 | 90.7 | 36.4 | 2.5 |
| Difenoconazole + Compound I | 2 + 5 | 63.3 | 39.1 | 1.6 |
| Difenoconazole + Compound I | 4 + 2.5 | 53.3 | 25.6 | 2.1 |
| Difenoconazole + Compound I | 2 + 2.5 | 43.3 | 28.9 | 1.5 |
| Azoxystrobin + Compound I | 0.8 + 10 | 80.0 | 64.0 | 1.3 |
| Azoxystrobin + Compound I | 0.4 + 10 | 83.3 | 31.0 | 2.7 |
| Azoxystrobin + Compound I | 0.2 + 10 | 61.7 | 22.0 | 2.8 |
| Azoxystrobin + Compound I | 0.8 + 5 | 80.0 | 66.8 | 1.2 |
| Azoxystrobin + Compound I | 0.4 + 5 | 83.3 | 36.4 | 2.3 |
| Azoxystrobin + Compound I | 0.2 + 5 | 63.3 | 28.1 | 2.3 |
| Azoxystrobin + Compound I | 0.8 + 2.5 | 80.0 | 61.2 | 1.3 |
| Azoxystrobin + Compound I | 0.4 + 2.5 | 80.0 | 25.6 | 3.1 |
| Azoxystrobin + Compound I | 0.2 + 2.5 | 66.7 | 15.9 | 4.2 |
| Pyraclostrobin + Compound I | 0.8 + 10 | 78.3 | 31.0 | 2.5 |
| Pyraclostrobin + Compound I | 0.4 + 10 | 70.0 | 26.5 | 2.6 |
| Pyraclostrobin + Compound I | 0.2 + 10 | 66.7 | 13.0 | 5.1 |
| Pyraclostrobin + Compound I | 0.8 + 5 | 53.3 | 36.4 | 1.5 |
| Pyraclostrobin + Compound I | 0.4 + 5 | 60.0 | 32.2 | 1.9 |
| Pyraclostrobin + Compound I | 0.2 + 5 | 66.7 | 19.8 | 3.4 |
| Pyraclostrobin + Compound I | 0.8 + 2.5 | 86.7 | 25.6 | 3.4 |
| Pyraclostrobin + Compound I | 0.4 + 2.5 | 65.0 | 20.8 | 3.1 |
| Pyraclostrobin + Compound I | 0.2 + 2.5 | 43.3 | 6.2 | 7.0 |
| Picoxystrobin + Compound I | 4 + 10 | 97.3 | 55.0 | 1.8 |
| Picoxystrobin + Compound I | 2 + 10 | 91.7 | 52.0 | 1.8 |
| Picoxystrobin + Compound I | 1 + 10 | 91.7 | 22.0 | 4.2 |
| Picoxystrobin + Compound I | 4 + 5 | 72.3 | 58.5 | 1.2 |
| Picoxystrobin + Compound I | 2 + 5 | 96.3 | 55.7 | 1.7 |
| Picoxystrobin + Compound I | 1 + 5 | 65.0 | 28.1 | 2.3 |
| Picoxystrobin + Compound I | 4 + 2.5 | 96.3 | 51.5 | 1.9 |
| Picoxystrobin + Compound I | 2 + 2.5 | 98.0 | 48.3 | 2.0 |
| Picoxystrobin + Compound I | 1 + 2.5 | 93.3 | 15.9 | 5.9 |
| Fluxapyroxad + Compound I | 0.8 + 10 | 46.7 | 19.0 | 2.5 |
| Fluxapyroxad + Compound I | 0.4 + 10 | 43.3 | 19.0 | 2.3 |
| Fluxapyroxad + Compound I | 0.2 + 10 | 53.3 | 31.0 | 1.7 |
| Fluxapyroxad + Compound I | 0.8 + 5 | 30.0 | 25.3 | 1.2 |
| Fluxapyroxad + Compound I | 0.8 + 2.5 | 33.3 | 12.7 | 2.6 |
| Fluxapyroxad + Compound I | 0.4 + 2.5 | 30.0 | 12.7 | 2.4 |
| Fluxapyroxad + Compound I | 0.2 + 2.5 | 33.3 | 25.6 | 1.3 |
| Benzovindiflupyr + Compound I | 4 + 10 | 58.3 | 23.5 | 2.5 |
| Benzovindiflupyr + Compound I | 2 + 10 | 53.3 | 13.0 | 4.1 |
| Benzovindiflupyr + Compound I | 1 + 10 | 30.0 | 16.0 | 1.9 |
| Benzovindiflupyr + Compound I | 4 + 5 | 50.0 | 29.5 | 1.7 |
| Benzovindiflupyr + Compound I | 2 + 5 | 56.7 | 19.8 | 2.9 |
| Benzovindiflupyr + Compound I | 1 + 5 | 53.3 | 22.5 | 2.4 |
| Benzovindiflupyr + Compound I | 4 + 2.5 | 46.7 | 17.6 | 2.7 |
| Benzovindiflupyr + Compound I | 2 + 2.5 | 38.3 | 6.2 | 6.1 |
| Benzovindiflupyr + Compound I | 1 + 2.5 | 26.7 | 9.5 | 2.8 |
| Penthiopyrad + Compound I | 2 + 10 | 28.3 | 19.0 | 1.5 |
| Penthiopyrad + Compound I | 1 + 10 | 25.0 | 13.0 | 1.9 |
| Penthiopyrad + Compound I | 4 + 5 | 61.7 | 41.9 | 1.5 |
| Penthiopyrad + Compound I | 2 + 5 | 33.3 | 25.3 | 1.3 |
| Penthiopyrad + Compound I | 1 + 5 | 33.3 | 19.8 | 1.7 |
| Bixafen + Compound I | 4 + 10 | 81.7 | 61.0 | 1.3 |
| Bixafen + Compound I | 2 + 10 | 86.7 | 46.0 | 1.9 |
| Bixafen + Compound I | 1 + 10 | 73.3 | 49.0 | 1.5 |
| Bixafen + Compound I | 4 + 5 | 92.3 | 64.0 | 1.4 |
| Bixafen + Compound I | 2 + 5 | 78.3 | 50.2 | 1.6 |
| Bixafen + Compound I | 1 + 5 | 65.0 | 53.0 | 1.2 |
| Bixafen + Compound I | 4 + 2.5 | 96.7 | 58.0 | 1.7 |
| Bixafen + Compound I | 2 + 2.5 | 93.3 | 41.8 | 2.2 |
| Bixafen + Compound I | 1 + 2.5 | 94.0 | 45.0 | 2.1 |
| Chlorothalonil + Compound I | 50 + 10 | 56.7 | 25.0 | 2.3 |
| Chlorothalonil + Compound I | 25 + 10 | 53.3 | 22.0 | 2.4 |
| Chlorothalonil + Compound I | 12.5 + 10 | 55.0 | 13.0 | 4.2 |
| Chlorothalonil + Compound I | 50 + 2.5 | 26.7 | 19.2 | 1.4 |
| Chlorothalonil + Compound I | 12.5 + 2.5 | 13.3 | 6.2 | 2.1 |
| Mancozeb + Compound I | 50 + 10 | 66.7 | 17.5 | 3.8 |
| Mancozeb + Compound I | 25 + 10 | 56.7 | 11.5 | 4.9 |
| Mancozeb + Compound I | 12.5 + 10 | 55.0 | 10.0 | 5.5 |
| Mancozeb + Compound I | 50 + 5 | 58.3 | 23.9 | 2.4 |
| Mancozeb + Compound I | 25 + 5 | 51.7 | 18.4 | 2.8 |
| Mancozeb + Compound I | 12.5 + 5 | 51.7 | 17.0 | 3.0 |
| Mancozeb + Compound I | 50 + 2.5 | 51.7 | 11.1 | 4.7 |
| Mancozeb + Compound I | 25 + 2.5 | 38.3 | 4.6 | 8.3 |
| Mancozeb + Compound I | 12.5 + 2.5 | 33.3 | 3.0 | 11.1 |

*ALTESO = Tomato early blight; Alternaria solani
*Observed = Observed percent disease control at the test rates
*Expected = Percent disease control expected as predicted by the Colby equation
*ppm = Parts per million
*Synergism factor = Observed/Expected

What is claimed:

1. A synergistic fungicidal mixture, comprising:
a fungicidally effective amount of the compound of Formula I, (S)-1,1-bis(4-fluorophenyl)propan-2-yl (3-acetoxy-4-methoxypicolinoyl)-L-alaninate: 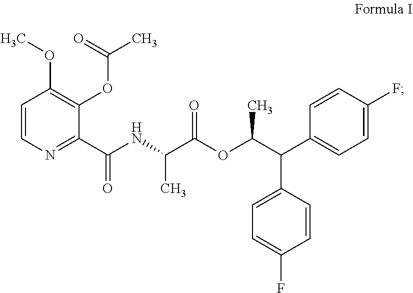 [SMILES: COc1ccnc(C(=O)N[C@@H](C)C(=O)O[C@@H](C)C(c2ccc(F)cc2)c2ccc(F)cc2)c1OC(C)=O]
and
at least one additional fungicide selected from the group consisting of sterol biosynthesis inhibitors, respiration inhibitors, and multi-site action inhibitors.

2. The mixture of claim 1 wherein the sterol biosynthesis inhibitor is selected from the group consisting of epoxiconazole, prothioconazole and difenoconazole.

3. The mixture of claim 2 wherein a concentration ratio of the compound of Formula I to epoxiconazole is from about 10:1 to about 1:1.6.

4. The mixture of claim 2 wherein a concentration ratio of the compound of Formula I to prothioconazole is from about 10:1 to about 1.3:1.

5. The mixture of claim 2 wherein a concentration ratio of the compound of Formula I to difenoconazole is from about 5:1 to about 1:1.6.

6. The mixture of claim 1 wherein the respiration inhibitor is selected from the group consisting of azoxystrobin, pyraclostrobin and picoxystrobin.

7. The mixture of claim 6 wherein a concentration ratio of the compound of Formula I to azoxystrobin is from about 50:1 to about 3.1:1.

8. The mixture of claim 6 wherein a concentration ratio of the compound of Formula I to pyraclostrobin is from about 50:1 to about 3.1:1.

9. The mixture of claim 6 wherein a concentration ratio of the compound of Formula I to picoxystrobin is from about 10:1 to about 1:1.6.

10. The mixture of claim 1 wherein the respiration inhibitor is selected from the group consisting of fluxapyroxad, benzovindiflupyr, penthiopyrad and bixafen.

11. The mixture of claim 10 wherein a concentration ratio of the compound of Formula I to fluxapyroxad is from about 50:1 to about 3.1:1.

12. The mixture of claim 10 wherein a concentration ratio of the compound of Formula I to benzovindiflupyr is from about 10:1 to about 1:1.6.

13. The mixture of claim 10 wherein a concentration ratio of the compound of Formula I to penthiopyrad is from about 10:1 to about 1.3:1.

14. The mixture of claim 10 wherein a concentration ratio of the compound of Formula I to bixafen is from about 10:1 to about 1:1.6.

15. The mixture of claim 1 wherein the multi-site inhibitor is selected from the group consisting of chlorothalonil and mancozeb.

16. The mixture of claim 15 wherein a concentration ratio of the compound of Formula I to chlorothalonil is from about 1:1.3 to about 1:20.

17. The mixture of claim 15 wherein a concentration ratio of the compound of Formula I to mancozeb is from about 1:1.3 to about 1:20.

18. The mixture of claims 1-17, wherein the mixture provides control of a fungal pathogen, and the pathogen is the causal agent of early blight of tomato (Alternaria solani).

19. A synergistic, fungicidal composition comprising of a fungicidally effective amount of the mixtures of claims 1-17 and an agriculturally acceptable carrier.
